(19) United States
(12) Patent Application Publication
(10) Pub. No.: US 2014/0343425 A1
(43) Pub. Date: Nov. 20, 2014

(54) ENHANCED ULTRASOUND IMAGING INTERPRETATION AND NAVIGATION

(71) Applicant: UNIVERSITY OF FLORIDA RESEARCH FOUNDATION, INCORPORATED, Gainesville, FL (US)

(72) Inventors: BARYS VALERIEVICH IHNATSENKA, Gainesville, FL (US); SAMSUN LAMPOTANG, Gainesville, FL (US); DAVID ERIK LIZDAS, Gainesville, FL (US); DIETRICH GRAVENSTEIN, Gainesville, FL (US); ANDRE BOEZAART, Gainesville, FL (US)

(73) Assignee: UNIVERSITY OF FLORIDA RESEARCH FOUNDATION, INCORPORATED, Gainesville, FL (US)

(21) Appl. No.: 14/276,858

(22) Filed: May 13, 2014

Related U.S. Application Data
(60) Provisional application No. 61/824,559, filed on May 17, 2013.

(51) Int. Cl. A61B 8/08; A61B 8/00

(52) U.S. Cl. CPC A61B 8/5292; A61B 8/4254
USPC 600/440; 600/443

(57) ABSTRACT

Systems and techniques are provided in which the orientation of an ultrasonic image display is configured to match the ultrasound probe orientation for ease of interpretation. The position and the orientation of an ultrasound probe are tracked using a tracking device having one or more position and orientation sensors. The orientation of a displayed ultrasound image is automatically adjusted to reflect the position and orientation of the ultrasound probe relative to the body structure being imaged. In some embodiments, a background image (with possible landmarks) is provided based on the location and orientation of the tracked ultrasound probe.

### CROSS-REFERENCE TO RELATED APPLICATION

The present application claims the benefit of U.S. Provisional Application Ser. No. 61/824,559, filed May 17, 2013, which is hereby incorporated by reference herein in its entirety, including any figures, tables, or drawings.

### BACKGROUND

Ultrasound imaging techniques that provide a real-time display of an imaged region are often used in operating room and diagnostic procedures. However, even if a clinician has a mental model of corresponding cross-sectional computed tomography (CT) or magnetic resonance imaging (MRI) image anatomy, it can sometimes be a challenge to match the structures on an ultrasound image with structures in the mental model. This occurs due to several reasons.

One reason is that the ultrasound window (width of the image) is much smaller than the usual CT slice. For example, the typical ultrasound window is about 38-60 mm depending on the footprint size and type of the ultrasound probe (e.g., curved or linear). This small size is effectively a tunnel-like image compared to the bigger picture available in CT and MRI. Furthermore, the depth of the picture is also generally limited to about 5-12 cm. Because the field of view from an ultrasound probe is limited, users may become disoriented, especially if they are used to larger images from CT and MRI. In other words, the ultrasound image may be too small to contain anatomical landmarks that would help the user in obtaining his or her bearings and interpreting the ultrasound image.

In addition to the small window and viewing depth, the viewing screen presents a fixed view. The fixed point of view of the ultrasound display screen is presented as if the body structure is being insonated from above; i.e., as if the probe is above the body structure. However, a hand-held ultrasound probe is freely moveable and can be placed on the body structure at any angle relative to the axis of the body structure, including from above, on the side on the side or at any other location relative to the structure. This can be seen in FIGS. 1A-1C. FIG. 1A is an ultrasound image as displayed on a view screen. However, as shown in FIG. 1B, a handheld probe may be used in a manner that the image is actually being taken from a side of a body structure (e.g., the thigh as shown in FIG. 1B). Thus, as shown in FIG. 1C, a practitioner makes a mental adjustment to have the ultrasound image reflect the direction of insonation.

Thus, if an anatomical structure is insonated from the side or from the bottom, a practitioner must do complex distracting; mental conversions/transformations/rotations of the image, and this is not easy and requires experience. This also makes ultrasound-guided needle placement more difficult and unnatural. Sometimes, clinicians will purposely tilt their head so that the ultrasound image display is “aligned” with the ultrasound probe orientation.

### BRIEF SUMMARY

Ultrasound imaging systems and techniques for facilitating interpretation of an ultrasound image are presented. In some embodiments, the orientation of an ultrasound image is automatically adjusted to correspond with a direction, orientation and/or position of insonation with respect to an anatomy. In some embodiments, background fill is provided based on the location and orientation of the ultrasound probe. In some further embodiments, the position of anatomical landmarks is recorded and displayed along with the ultrasound image.

By displaying an ultrasound image with features including one or more of correlated orientation, background fill, and anatomical landmarks, an ultrasound imaging system of certain embodiments of the invention can contribute to increased productivity, efficiency and accuracy while minimizing risk of misinterpretation, simplifying interpretation and promoting patient safety. Certain embodiments facilitate interpretation of, and navigation using, ultrasound images.

According to one implementation, orientation and positioning sensor data received from an ultrasound probe are used to automatically orient the displayed ultrasound image. In some implementations, the background of the display is filled in to provide context and landmarks. In a further embodiment, the background is automatically filled and aided navigation is provided.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1A is an ultrasound image as displayed on a view screen of a typical scenario.

FIG. 1B illustrates an ultrasound probe being used at a side of a body structure.

FIG. 1C illustrates a realignment of the displayed ultrasound image based on the actual ultrasound probe position shown in FIG. 1B.

FIG. 2 illustrates an operating environment in which an embodiment may be implemented.

FIG. 3 illustrates an ultrasound probe that may be used in an implementation shows an ultrasound probing system according to one embodiment.

FIG. 4 illustrates a diagram of an ultrasound image processing unit.

FIG. 5 illustrates a process flow according to an embodiment.

FIGS. 6A-6C illustrate a display of the process flow according to an embodiment.

### DETAILED DESCRIPTION

Ultrasound imaging systems and techniques for facilitating interpretation of an ultrasound image are presented. In some embodiments, the orientation of an ultrasound image is automatically adjusted to correspond with a direction, orientation and/or position of insonation with respect to an anatomy. In some embodiments, background fill is provided based on the location and orientation of the ultrasound probe. In some further embodiments, the position of anatomical landmarks is recorded and displayed along with the ultrasound image.

A system is provided in which the orientation of an ultrasonic image display is configured to match the ultrasound probe orientation for ease of interpretation. According to one embodiment, the position and the orientation in 3D space of a handheld ultrasound probe are tracked using one or more position and orientation sensors. The orientation of the ultrasound image displayed on a monitor (or display) is automatically adjusted to reflect the position and orientation of the ultrasound probe relative to the body structure being imaged. In one implementation, the one or more position and orientation sensors are provided in the form of a six-degrees of freedom (6-DOF) tracker. The position and orientation sensor(s) may be a magnetic tracker.

The background image for the oriented ultrasound image can be selected by the user or automatically applied to provide context and landmarks. In some cases, a background is filled in for missing areas of the image. A missing area of an image refers to the black regions generally found on the display of an ultrasound image, for example the regions outside the view window or of material that does not show up in the ultrasound image.

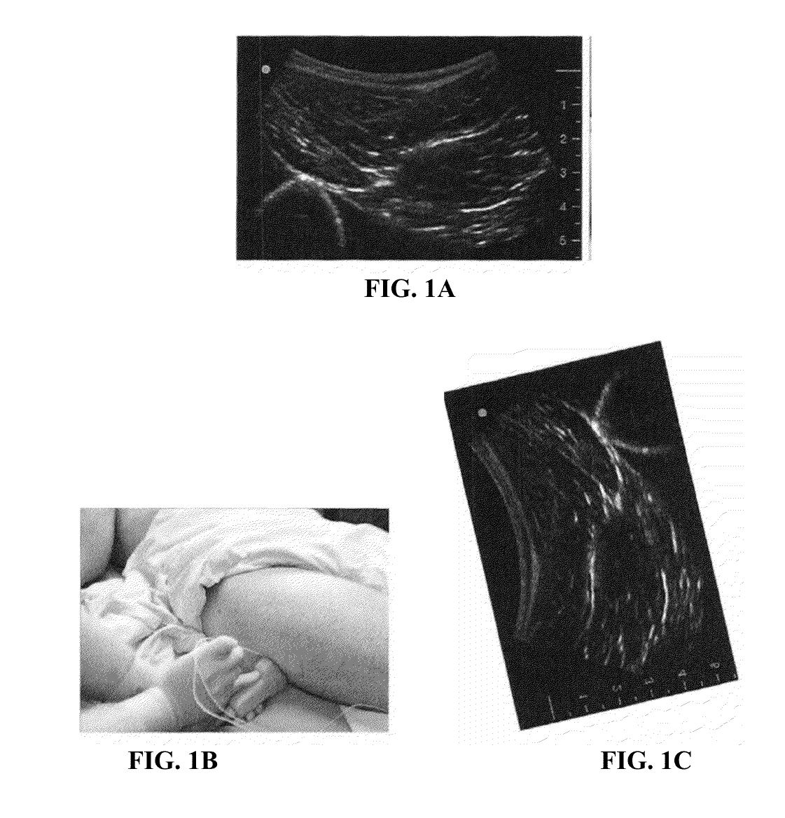

According to an embodiment, the missing background (e.g., black regions at the top right and top left of the ultrasound image of FIG. 1A) is filled in using a pre-recorded background that could be, among others, a virtual rendition, a CT scan image, a MRI scan image, or a positron emission tomography (PET) image. The pre-recorded background can be selected from a set of backgrounds corresponding to different slices of different body parts.

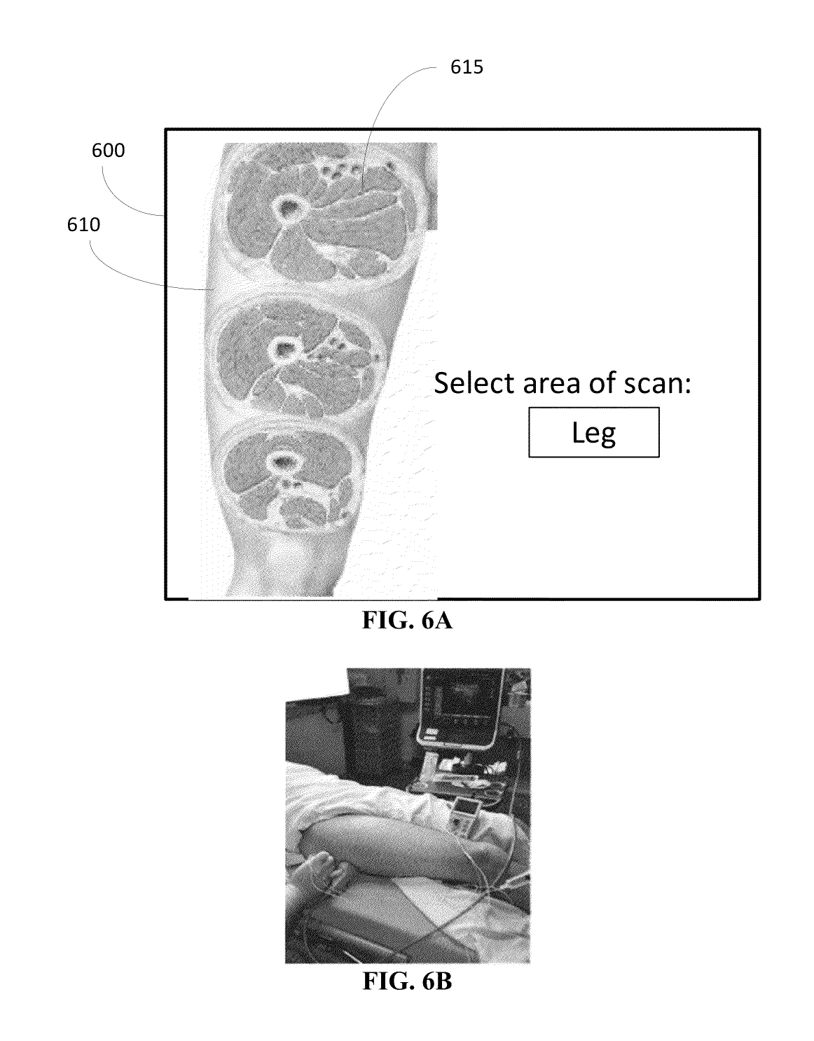

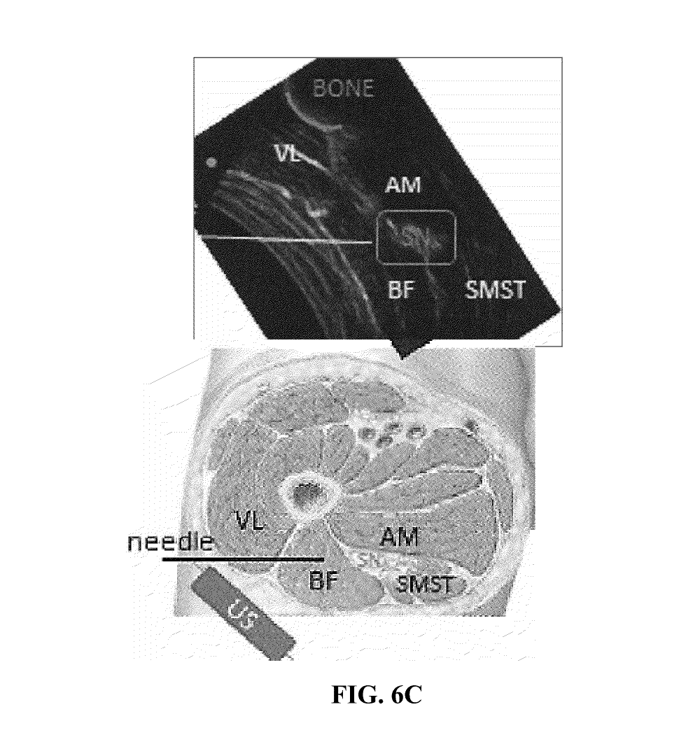

When imaging a subject, the user can select a background for filling the image. The background image can be selected through a user interface displaying a representation of a body. When the system receives a selection through the user interface, a listing or thumbnails or other display of slices that may be associated with that selection may be displayed. In some cases, there are multiple types of background images of varying detail that may be available for a particular body part selection. The user can, for example, select the slice to be used to fill in the missing background by selecting a probe position on an icon of a human body (see e.g., FIG. 6A).

As mentioned above, a user can select the background for the image. However, the pre-recorded images available for selection may be of a normal sized person, or the available images for selection may not include a background that corresponds to certain characteristics of the subject. For example, the pre-recorded backgrounds may be of a normal sized person while the actual patient may be heavy set or petite to an extent that may lead to some incongruence between the ultrasound image and the pre-recorded background images.

Accordingly in another embodiment, additional tracking sensors can be provided, for example as navigation pads located on at least two known external anatomical landmarks such as the sternal notch and the hip bone. The distance between two landmarks can be measured and an approximation of the body type of the patient can be made. Using the determination of the patient body type, the background can be filled automatically with a suitably sized image and navigation can be aided.

The knowledge of patient body type (and size) allows (a) automated selection of the pre-recorded slice based on the tracked probe position relative to the anatomical landmark trackers, (b) automatic scaling up or down of the pre-recorded backgrounds to match patient size and (c) adjustments in response to any shifts in patient body position that are detected by the anatomical landmark trackers. The automated selection of pre-recorded slice based on the traced probe position uses three or more tracking sensors.

In one embodiment, a single tracked sensor may be used on the ultrasound probe. The tracked ultrasound probe can be used to locate at least two anatomical landmarks. The two detected landmarks can be recorded and used in the system's algorithms, and each anatomical landmark can be confirmed (e.g., by comparison to standard views). According to one such implementation, the tracking sensor in the probe can be used to record landmarks that the system directs the user to locate. For example, a software program running as part of the system may prompt the user to place the probe at location A and then to location B, recording the readings at each location to obtain the distance between locations A and B and, thus, an estimate of the body size.

This approach of measuring the inter-anatomical landmark distance and having a tracked ultrasound probe could also help in navigation by labeling 3D regions where certain structures such a brachial plexus, axillary vein are most likely to be located based on the known body size, the known location of the anatomical landmarks and the known location of the tracked ultrasound probe.

A greater understanding of the present invention and of its many advantages may be had from the following examples, given by way of illustration. The following examples are illustrative of some of the methods, applications, embodiments and variants of the present invention. They are, of course, not to be considered in any way limitative of the invention. Numerous changes and modifications can be made with respect to the invention.

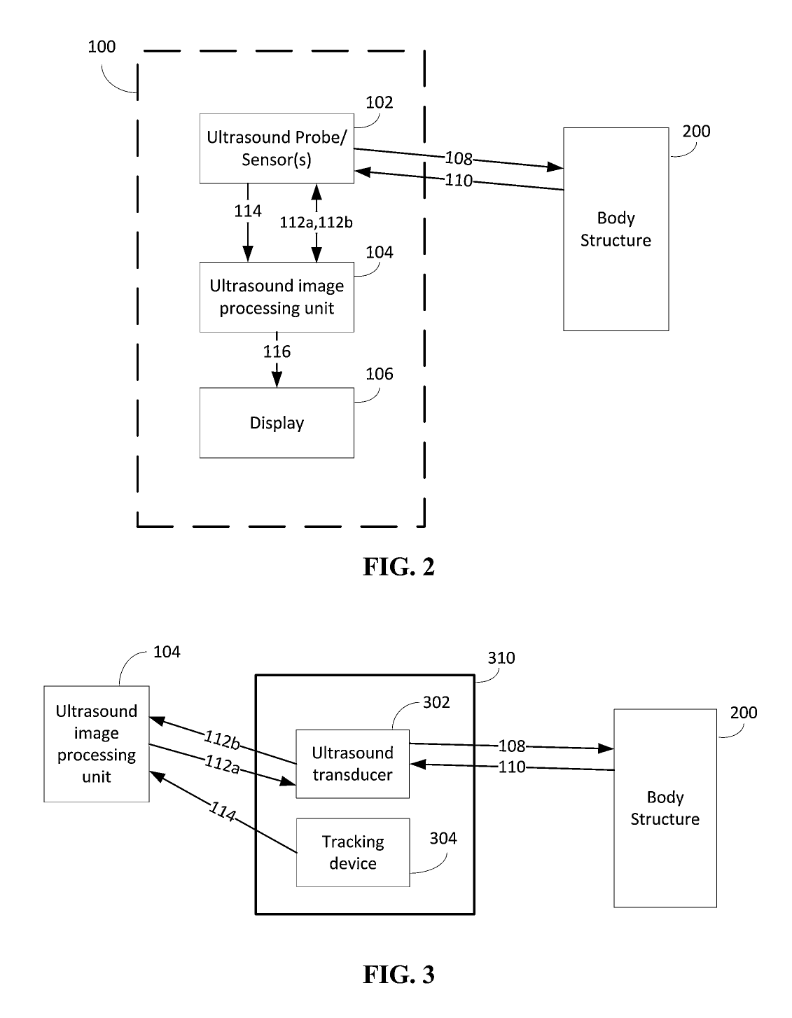

FIG. 2 illustrates an operating environment in which an embodiment may be implemented. Referring to FIG. 2, an ultrasound imaging system 100 can include an ultrasound probe 102 with one or more sensors for providing position and orientation information about the ultrasound probe 102 to an ultrasound image processing unit 104 in order to provide an oriented image for viewing in a display 106. The ultrasonic probe 102 includes a transducer in order to transmit acoustic waves 108 and receive reflected acoustic waves 110. The ultrasound image processing unit 104 and the ultrasonic probe 102 communicate with each other to transmit and receive signals 112a, 1112b.

The ultrasound image processing unit 104 provides control signals 112a to the ultrasound probe 102 to form the acoustic waves 108 and receives the electrical pulses 1121 created from the reflective acoustic waves 110 to process the data and generate an image for display. The ultrasound image processing unit 104 can be configured to receive signals 114 from the one or more sensors attached to the ultrasound probe 102 and use the detected orientation and position of the probe to orient the ultrasound image 116 displayed at the display 106.

In operation, an ultrasound image can be obtained of a body structure 200 by receiving the reflective acoustic waves 110 and combining the signal from the ultrasound probe 102 with the signal from the position and orientation sensor(s) to generate an oriented ultrasound image for display.

FIG. 3 illustrates an ultrasound probe that may be used in an implementation. Referring to FIG. 3, an ultrasound probe can include the transducer 302 and a tracking device 304. The tracking device 304 may include one or more sensors such as a 6-DOF sensor that measures displacement and orientation in three-dimensional space. The transducer 302 and tracking device 304 (e.g., 6-DOF magnetic sensor, or other sensors providing similar functionality) may be encased in a housing 310 of the probe or the transducer 302 is encased in the housing 310 while the tracking device 304 is attached on or within a portion of the housing 310; both scenarios enabling the transducer and sensor(s) to move as one unit during scanning sessions of a body structure 200.

The tracking device 304 measures and tracks position and orientation of the ultrasound transducer 302 and informs the ultrasound image processing unit 104 about the position (x, y, z coordinates) and the orientation (pitch, yaw, roll) of the ultrasound transducer 302 by transmitting a probe tracking signal 114 to the ultrasound image processing unit 104 throughout the scanning session.

Prior to an acquisition of tracking data, the tracking device 304 may be calibrated. Calibration may include determining offsets of position and/or orientation of tracking sensors of the tracking device 304. The calibration data of the tracking sensors are transmitted from the tracking device 304 to the ultrasound image processing unit 104. The tracking sensors may be calibrated initially, prior to and/or during a scanning procedure.

Measured positions and orientations of the ultrasound transducer 302 (and probe) depend on the sensed positions and orientations of ultrasound transducer 302 and the calibration data of the tracking sensors. The ultrasound image processing unit 104 can be configured to interpret the signals and determine the position and orientation of the probe in order to perform additional processes for displaying the ultrasound image.

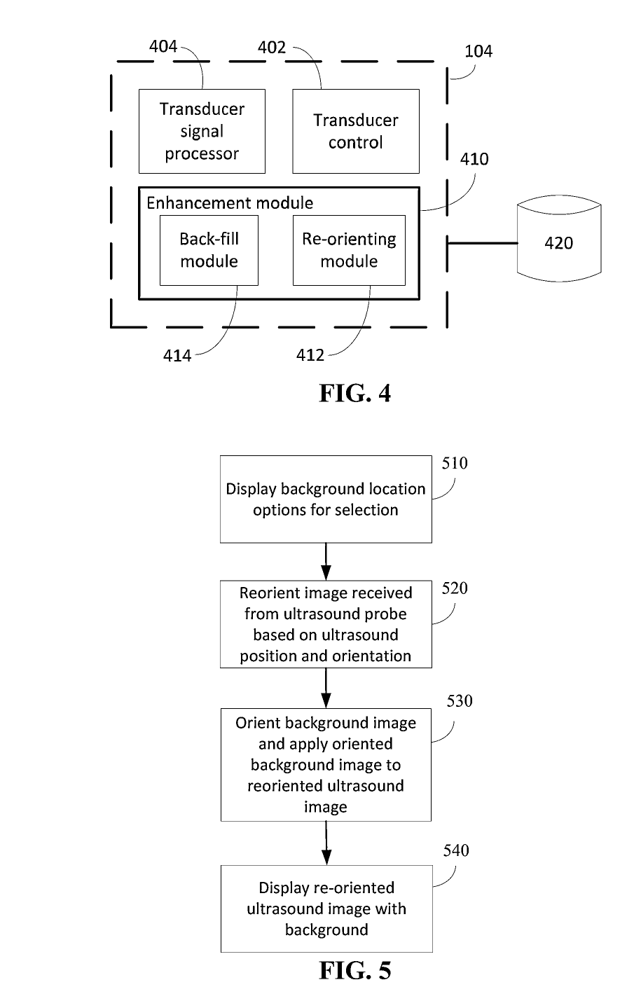

FIG. 4 illustrates a diagram of an ultrasound image processing unit. Referring to FIG. 4, the ultrasound image processing unit 104 can include a transducer control 402 that provides the control signals 112a to the ultrasound probe 102 and a transducer signal processor 404 that receives the signals 112b from the ultrasound probe 102 to generate an ultrasound image. According to embodiments of the invention, the ultrasound image processing unit 104 can include an enhancement module 410 that can include a re-orienting module 412 that receives the signals 114 from the tracking device (e.g., the one or more sensors) to orient the ultrasound image according to how the probe is positioned, a back-fill module 414 that provides a background to the ultrasound image, or both. A memory 420 can be included as part of or in communication with the processing unit 104.

The re-orienting module 412 can determine the orientation and position of the ultrasound transducer from the received signals from the tracking device, which indicate the rotation and/or movement of the ultrasound probe.

The re-orienting module 412 can be configured to orient the ultrasound image generated by the transducer signal processor 404 (a “pre-processed ultrasound image”) by matching the coordinate system of the ultrasound probe determined using the signals from the tracking device to the coordinate system of the pre-processed ultrasound image (from the transducer signal processor 404), and then translating the orientation and position view of the pre-processed ultrasound image based on the relative orientation and position of the ultrasound probe. The pre-processed ultrasound image may be in two-dimensional or three-dimensional format.

The position and orientation of the ultrasound images can be determined based on one or more points of fixed position of the ultrasound probe. Once the point(s) of fixed position(s) of the ultrasound probe is determined, this information may be stored in the memory 420. The point of fixed position of the ultrasound probe can correspond to the location where the tracking device is attached to the instrument. In embodiments where the point of fixed position used by the re-orienting module 412 is in a different location, an additional step of deriving the spatial positional information of the point fixed to the ultrasound probe from other fixed points may be performed.

With this information, the re-orienting module 412 can map each of the six coordinates including x, y, z, yaw, pitch, and roll of the ultrasound probe onto the coordinates/position of the pre-processed ultrasound image.

By performing coordinate mapping, a processed image can be generated from a view related to the spatial position of the ultrasound probe. The orientation and position mapping information can be used to translate the orientation and position of the ultrasound image (e.g., move and rotate the ultrasound image) to correspond the orientation and position of the ultrasound image to the orientation and position of the ultrasound probe. This translated image can be provided as the processed image signal.

In some cases, the re-orienting module 412 performs a method in which a decision is made as to whether or not the unprocessed ultrasound image should be rotated and/or moved due to the detection signals from the tracking device. If it is determined that the ultrasound probe is moved and/or rotated, then the orientation and position of the ultrasound image is translated (e.g., moved and/or rotated) according to the changed orientation and position to generate a processed image signal for display.

The processed image signals can be two-dimensional images along planes that are trans-axial or orthogonal to the position of the ultrasound probe. The processed image signals can also be three-dimensional projection images.

In either case, the processed image signals represent images of a body structure from the view of the ultrasound probe.

The back-fill module 414 can be configured to fill in the ultrasound images to provide context and landmarks. When displaying an acquired ultrasound image, at least one of a virtual representation, CT image, a MRI image, and/or PET image can be provided as background for the ultrasound image. The background fill image(s) can be stored in the memory 420. In one embodiment, a user can select a pre-recorded background image. A user interface, e.g., a graphical user interface (GUI), can be provided for facilitating selection and viewing of an ultrasound image (with background) to interactively view and/or manipulate the combined images.

In certain embodiments, the background images stored in the memory for use as a background can be selected from a set of backgrounds corresponding to different slices of different body parts. The user could for example select the slice to be used to fill in as background by selecting a probe position or a standard cross-sectional slice on an icon of a human body. FIG. 6A illustrates an example representation of an interface in which a user can select a region that will be scanned for use as a back-ground fill for the ultrasound image when scanning a leg as shown in FIG. 6B. In another embodiment, the background image can be selected automatically by the system through use of image recognition or additional tracking devices. In cases where the patient stays immobile, the background image can be selected automatically through the use of the real-time position and orientation of the ultrasound probe relative to two known landmarks whose coordinates were previously acquired by, when prompted, placing the ultrasound probe on these landmarks.

In some cases, the background can fill in areas of the image that do not contain areas constructed from the transducer signals (either because there was not a feature that would show in an ultrasound image or because of the re-orientation of the image changing how the image fills the display). In some cases, in addition or as an alternative, the background image may provide a graphical (or virtual) representation, CT image, MRI image, PET image or other helpful image of a tissue or context of a tissue being imaged.

The available background images may be stored in a database associated with the ultrasound imaging system (e.g., memory 420) and/or may be acquired from an external source (including from the Web). The images may include images corresponding to differently sized patients, for example based on sex, height, age, weight, and the like. The granularity and correspondence of the various images to a patient's sex, height, age, weight, and the like may vary in different implementations. In some cases, a few options are available. In other cases, a closer match to the patient may be available.

The background images may be re-sized and combined with (fused) or overlaid (or back-filled) on an ultrasound image (which can be part of the functionality of the back-fill module 414) to enhance the visualization by a user.

For embodiments performing an automatic selection of a background image, navigation pads containing a tracking sensor can be used to detect and measure distances among established two or more points of anatomical landmarks. The distance(s) between certain anatomical landmarks can be used to calculate an approximate body type/size, which can be used to select a size adjusted image slice as a background. This embodiment can be applicable when the patient is not immobile during imaging.

The navigation pads (each containing a tracking sensor) can be attached on at least two known external anatomical landmarks such as the sternal notch and the hip bone so that the distance between the two landmarks can be measured and an approximation of the body type of the patient can be made. This knowledge allows automated selection of the pre-recorded slice based on the tracked probe position relative to the anatomical landmark trackers, automatic scaling up or down of the pre-recorded backgrounds to match patient size and adjustments in response to any shifts in patient body position that are detected by the anatomical landmark trackers. This approach would require at least three tracking sensors.

Automated selection of structure may include a combined coarse and fine search for pre-recorded images of body structure. For example, a less accurate initial position or range of positions for a given body structure is determined first. This position is then refined. The coarse positioning and/or the refined position may use machine-trained classifiers. The positions of other structure may be used in either coarse or fine positioning. The structure or structures may be identified without user input.

FIG. 5 illustrates a process flow according to an embodiment; and FIGS. 6A-6C illustrate a display-flow according to an embodiment. According to certain scenarios, an ultrasound imaging system can display background location options for selection (510). For example, referring to FIG. 6A, a user interface 600 may be displayed in which a user can select a body part area of where a scan will be (or is) taking place (e.g., as shown in FIG. 6A). In the example shown in FIG. 6A, a leg 610 is selected and various available regions 615 (and corresponding slices) are available for selection. Returning to FIG. 5, upon receiving the signals (and/or generated image) from the ultrasound transducer during scanning, the system can reorient the image received from the ultrasound probe based on the ultrasound probe position and orientation (520). When a background is selected from user interface 600 (or upon automatic determination of a corresponding background), the background image can be oriented to match the re-oriented ultrasound image and applied as back-fill, overlay, schematic overlay, abstracted overlay, label overlay, side-by-side or other visual aid (530) and then displayed to the user (540). FIG. 6C shows oriented images of the ultrasound and background that may be combined for display.

In some cases, operation 520 may be omitted or replaced with a process that re-orients (or rotates) the monitor or screen displaying the ultrasound image. For example, the monitor or screen displaying the ultrasound image can be mechanically rotated (manually or automatically) within the plane of the monitor until the displayed ultrasound image (which stays fixed relative to the monitor) is aligned with the ultrasound probe orientation.

In some cases, a mismatch of the ultrasound image and background/overlay may occur. In such cases, an alarm or error message may be provided. In one implementation, when a user selects an incorrect background image, the system may recognize from the ultrasound image that the background does not match and a prompt may be provided for the user to select a different background image.

Certain techniques set forth herein may be described or implemented in the general context of computer-executable instructions, such as program modules, executed by one or more computing devices. Generally, program modules include routines, programs, objects, components, and data structures that perform particular tasks or implement particular abstract data types.

Embodiments may be implemented as a computer process, a computing system, or as an article of manufacture, such as a computer program product or computer-readable medium. Certain methods, processes, and modules described herein can be embodied as code and/or data, which may be stored on one or more computer-readable media. Certain embodiments of the invention contemplate the use of a machine in the form of a computer system within which a set of instructions, when executed, can cause the system to perform any one or more of the methodologies discussed above. Certain computer program products may be one or more computer-readable storage media readable by a computer system and encoding a computer program of instructions for executing a computer process.

Computer-readable media can be any available computer-readable storage media or communication media that can be accessed by the computer system.

Communication media include the mechanisms by which a communication signal containing, for example, computer-readable instructions, data structures, program modules, or other data, is transmitted from one system to another system. The communication media can include guided transmission media, such as cables and wires (e.g., fiber optic, coaxial, and the like), and wireless (unguided transmission) media, such as acoustic, electromagnetic, RF, microwave and infrared, that can propagate energy waves. Communication media, particularly carrier waves and other propagating signals that may contain data usable by a computer system, are not included as computer-readable storage media.

By way of example, and not limitation, computer-readable storage media may include volatile and non-volatile, removable and non-removable media implemented in any method or technology for storage of information such as computer-readable instructions, data structures, program modules or other data. For example, a computer-readable storage medium includes, but is not limited to, volatile memory such as random access memories (RAM, DRAM, SRAM); and non-volatile memory such as flash memory, various read-only-memories (ROM, PROM, EPROM, EEPROM), magnetic and ferromagnetic/ferroelectric memories (MRAM, FeRAM), and magnetic and optical storage devices (hard drives, magnetic tape, CDs, DVDs); or other media now known or later developed that is capable of storing computer-readable information/data for use by a computer system. “Computer-readable storage media” do not consist of carrier waves or propagating signals.

In addition, the methods, processes, and modules described herein can be implemented in hardware modules. For example, the hardware modules can include, but are not limited to, application-specific integrated circuit (ASIC) chips, field programmable gate arrays (FPGAs), and other programmable logic devices now known or later developed. When the hardware modules are activated, the hardware modules perform the methods and processes included within the hardware modules.

Any reference in this specification to “one embodiment,” “an embodiment,” “example embodiment,” etc., means that a particular feature, structure, or characteristic described in connection with the embodiment is included in at least one embodiment of the invention. The appearances of such phrases in various places in the specification are not necessarily all referring to the same embodiment. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

It should be understood that the examples and embodiments described herein are for illustrative purposes only and that various modifications or changes in light thereof will be suggested to persons skilled in the art and are to be included within the spirit and purview of this application.

What is claimed is:

1. An ultrasound imaging system comprising:
an ultrasound probe;
a tracker device attached to the ultrasound probe, the tracker device comprising one or more sensors for detecting position and orientation of the ultrasound probe; and
a processing unit configured to receive sensor signals from the tracker device; determine position and orientation of the ultrasound probe using the sensor signals; and orient an ultrasound image generated from ultrasound signals received from the ultrasound probe based on the position and orientation of the ultrasound probe.

2. The ultrasound imaging system of claim 1, further comprising:
a database of background images,
wherein the processing unit is further configured to receive a selection of a background image from the database of background images; orient the background image based on the position and orientation of the ultrasound probe; and apply the background image to the ultrasound image for display.

3. The ultrasound imaging system of claim 2, further comprising:
one or more navigation pads each incorporating a tracking sensor,
wherein the processing unit is further configured to receive landmark signals from the one or more navigation pads; determine a region being scanned by using the landmark signals; and automatically select the background image from the database of background images.

4. The ultrasound imaging system of claim 3, wherein the processing unit is further configured to calculate approximate size of patient from the landmark signals; and determine an appropriately sized image slice for the background image.

6. The ultrasound imaging system of claim 5, wherein determining the appropriately sized image slice comprising scaling an image from the database of background images based on approximate size of the patient.

7. The ultrasound imaging system of claim 2, wherein the processing unit is further configured to initiate prompts to a user to record one or more anatomical landmarks; acquire the coordinates of the one or more anatomical landmarks; determine a region being scanned by using the coordinates of the one or more anatomical landmarks as a reference for a position of the probe relative to the body; and automatically select the background image from the database of background images.

8. The ultrasound imaging system of claim 7, wherein the processing unit is further configured to calculate approximate size of patient from the coordinates of the one or more anatomical landmarks; and determine an appropriately sized image slice for the background image.

9. The ultrasound imaging system of claim 8, wherein determining the appropriately sized image slice comprising scaling an image from the database of background images based on approximate size of the patient.

10. The ultrasound imaging system of claim 2, wherein the processing unit is further configured to display a user interface from which the background image is selected.

11. The ultrasound imaging system of claim 2, wherein the background image comprises a virtual representation, a computed tomography (CT) image, a magnetic resonance imaging (MRI) image, or a positron emission tomography (PET) image.

12. An ultrasound imaging system comprising:
a database of background images; and
a processing unit configured to receive a selection of a background image from the database of background images; orient the background image based on a position and orientation of an ultrasound probe; and apply the background image to an ultrasound image for display.

13. A system comprising one or more computer-readable storage media having instructions, that when executed by a processing system, direct the processing system to:
determine a location and orientation of a tracked imaging probe with respect to a body by reference to the location and orientation of one or more anatomical landmarks.

14. The system of claim 13, further comprising:
one or more navigation pads each incorporating a tracking sensor,
wherein the instructions to determine the location and orientation of the tracked imaging probe, direct the processing system to receive landmark signals from the one or more navigation pads; and determine a region being scanned by using the landmark signals.

15. The system of claim 13, wherein the instructions to determine the location and orientation of the tracked imaging probe direct the processing system to:
prompt a user to use a tracked ultrasound probe to acquire the coordinates of one or more anatomical landmarks; and
determine a position of the ultrasound probe relative to the body using the acquired one or more anatomical landmarks.

16. A method for enhancing ultrasound imaging interpretation and navigation, the method comprising:
receiving sensor signals from a tracker device associated with an ultrasound probe;
determining position and orientation of the ultrasound probe using the sensor signals; and
orienting an ultrasound image generated from ultrasound signals received from the ultrasound probe based on the position and orientation of the ultrasound probe.

17. The method of claim 16, further comprising:
receiving a selection of a background image from a database of background images;
orienting the background image based on the position and orientation of the ultrasound probe; and
applying the background image to the ultrasound image for display.

18. The method of claim 17, further comprising:
receiving landmark signals from one or more navigation pads;
determining a region being scanned by using the landmark signals; and
automatically selecting the background image from the database of background images.

19. The method of claim 17, further comprising:
initiating prompts to a user to record one or more anatomical landmarks;
acquiring the coordinates of the one or more anatomical landmarks;
determining a region being scanned by using the coordinates of the one or more anatomical landmarks as a reference for a position of the ultrasound probe relative to the body; and
automatically select the background image from the database of background images.

20. The method of claim 17, further comprising:
displaying a graphical user interface from which the background image is selected.
